(19) United States
(12) Patent Application Publication
(10) Pub. No.: US 2023/0201557 A1
(43) Pub. Date: Jun. 29, 2023

(54) INFUSION PUMP FOR IDENTIFYING THE COLOR OF A HOSE CLAMP WITH IR MARKERS

(71) Applicant: B. Braun Melsungen AG, Melsungen (DE)

(72) Inventors: Andreas Katerkamp, Melsungen (DE); Markus Discher, Knuellwald (DE); Christoph Erlen, Kassel (DE); Maria Buerger, Malsfeld (DE); Jan Schwarz, Melsungen (DE); Mario Richardt, Zierenberg (DE); Stephan Hoevel, Kassel (DE); Jan Fragner, Kassel (DE); Jan Eric Herr, Kassel (DE); Volker Schumacher, Bad Waldsee (DE); Simon Paul, Biberach (DE); Raphael Jedelhauser, Langenau (DE)

(21) Appl. No.: 18/088,922

(22) Filed: Dec. 27, 2022

(30) Foreign Application Priority Data
Dec. 28, 2021 (DE) 10 2021 215 067.5

(51) Int. Cl. A61M 39/28; A61M 5/142

(52) U.S. Cl. CPC A61M 39/284; A61M 5/142; A61M 2205/6081; A61M 2205/6045

(57) ABSTRACT

A clamping module of or for a medical pump, and a method for identifying a hose clamp of a disposable infusion article insertable into the clamping module. The clamping module includes an electro-optical system for identifying the hose clamp, which has an optical marking. The electro-optical system includes a first light source for emitting at least a first light beam and a second light source for emitting at least a second light beam. The second light source is formed differently than the first light source. A photodetector receives a reflected light, and an optical window bundles the first light beam and/or the second light beam on the disposable infusion article, as well as the reflected light received by the photodetector. The method identifies the hose clamp based on a color and uses a measurement algorithm after insertion of the hose clamp into the medical pump.

### CROSS-REFERENCE TO RELATED APPLICATIONS

This application claims priority under 35 U.S.C. § 119 to German Application No. 10 2021 215 067.5, filed Dec. 28, 2021, the content of which is incorporated by reference herein in its entirety.

### FIELD

The present disclosure relates to a clamping module/ clamp module of or for a medical pump, in particular an infusion pump, comprising an electro-optical system for identifying a hose clamp of a disposable infusion article insertable into the clamping module, and thus determining the type of disposable infusion article. Furthermore, the present disclosure relates to a method for identifying a hose clamp based on color via a measurement algorithm after insertion of the hose clamp into a medical pump.

### BACKGROUND

Infusion pumps and infusion sets often include a slide clamp/ hose clamp. In this regard, the hose clamp is part of the infusion set and interacts with the hose of the infusion set to selectively prevent free flow through the infusion hose. Often, such a hose clamp is actuated by the pump itself during an insertion process of the infusion set into the pump, for example via a front flap of the pump during its closing movement. Alternatively, the hose clamp may also be manually actuated independently of any closing mechanisms on the pump.

The hose clamp typically has a tapered elongated hole through which the hose passes. The hose is not closed at one end of the hole and is compressed when pushed to the other end of the hole. In some cases, the hose clamp, designed as a slide clamp, functionally serves as a key to turn the infusion pump on and off and may help secure the hose of the infusion set in the pump channel. On some pumps, the hose clamp/ slide clamp also serves as a key to open the pump door to load or unload the infusion hose. The pump door is unlocked by pushing part of the hose clamp/ slide clamp into a slot in the pump body. Pressing the hose clamp/ slide clamp into the slot pushes the hose to the narrow end of the conical hole, thus closing the hose. Thus, the hose clamp provides a measure of safety by preventing free flow, since it compresses the hose when the pump door is opened.

Sliding/slide clamps of this type are often designed to fit a specific pump, and thus ensure that only compatible infusion sets are used with that pump. Against this background, it is important to be able to ensure that the correct hose clamp (sliding/slide clamp) is used or, respectively, to be able to identify which type of infusion hose set is used.

U.S. Pat. No. 9,272,129 B2 is known from the prior art in which an infusion set is provided in which a slide clamp/ hose clamp can be coded by the use of colors, holes or the like to identify the type of infusion hose used therewith. The infusion pump can identify the code on the hose clamp/ slide clamp and thereby determine the type of infusion hose, thereby allowing access only to infusion programs for drugs or infusion solutions compatible with the particular infusion hose type.

In addition, a system and method for automatically administering an infusate to a patient is disclosed in US 2006/0224128 A1. The system includes an infusion set and an infusion device. A signaling component disposed on an infusion set component identifies an administration protocol for the infusion set. A sensing device operatively connected to the infusion device senses the signaling component and identifies the administration protocol. The infusion device is then configured to operate in accordance with the administration protocol.

However, the prior art has the disadvantage that the color identifying of the hose clamp is inadequate, since color spectra cannot be separated sufficiently with pure RGB illumination.

### SUMMARY

Against this background, the present disclosure is based on the objectively technical object of eliminating or at least reducing the disadvantages of the prior art, in particular to provide a medical pump which enables reliable detection of an inserted hose clamp/ slide clamp.

Therefore, the present disclosure relates to a clamping module of or for a medical pump, in particular an infusion pump, which has or forms an electro-optical system for identifying a hose clamp of a disposable infusion article insertable into the clamping module, in particular an infusion hose, wherein the hose clamp has a predetermined color and/or an optical/optically identifiable marking. The clamping module has a first light source for transmitting at least a first light beam, a second light source for transmitting at least a second light beam, wherein the second light source is formed differently from the first light source, or the frequencies/wavelengths of the at least two light beams are different from each other, a photodetector for receiving reflected light, and an optical window for focusing the at least first light beam and/or the at least second light beam on the disposable infusion article as well as the reflected light in the photodetector.

In other words, the present disclosure relates to a clamping module of a medical pump, in particular an infusion pump, with an electro-optical system for identifying the color/color marking of a hose clamp of a disposable infusion article and for controlling the medical pump/infusion pump accordingly. The hose clamp inside the medical pump/infusion pump is illuminated with different light sources/different wavelengths and the color of the hose clamp is determined via the reflected light and the type of disposable infusion article is identified in this way.

When the disposable infusion article is inserted, the disposable infusion article is identified by a sensor and a control unit in the infusion pump via the identifying feature and used to activate parameters in the control unit of the infusion pump. These parameters are for example
parameters for controlling the identification of pressure,
parameters for controlling identification of air,
parameter for defining the maximum dwell time of the disposable infusion article, and/or
parameters for air-stop filter (yes/no).

The parameters to be activated can be stored in a disposable-article data set. The pump allows the user to define and monitor the infusion via the input device and the display. In other words, this means an expansion of the spectral space with optically similar color spectra.

The present disclosure provides the advantages or improvements of making the color of the hose clamp/slide clamp usable/identifiable by both the user/operator and the machine. Thus, the present disclosure provides machine readability and user readability simultaneously. The preferably additional optical marking allows an imaging space to be expanded and the colors to be separated accordingly.

It is preferred if the first light source is an RGB LED, preferably a combined RGB LED, and the second light source is an IR LED, in particular with a wavelength of 950 nm±42 nm, wherein the first light source comprises three LEDs which are configured to output the first light beam as a red, in particular with a wavelength of 630 nm±16 nm, a green, in particular with a wavelength of 520 nm ±33 nm, and/or a blue, in particular with a wavelength of 467 nm±25 nm, first light beam.

In other words, the electro-optical system inside the medical pump or, respectively, inside the clamping module that is inserted/insertable into the medical pump has or forms the combined RGB LED, the IR LED, the photodetector and the optical window for focusing the light beam on the hose clamp as well as the reflected light into the photodetector. Therefore, for differentiated color identifying, RGB signals and IR signals are used to illuminate the hose clamp.

Accordingly, the following wavelengths are used in the present disclosure:

Red: main wavelength 630 nm±16 nm

Green: main wavelength 520 nm±33 nm

Blue: main wavelength 467 nm±25 nm

Infrared (IR): main wavelength 950 nm±42 nm

With regard to the materials used, this results in the following overview (lookup table) of the material properties. These proportions depend on the material. It is therefore obvious that the proportions change when other materials are used. In other words, depending on the material used and the color/pigment admixture of the clamp, the irradiated main wavelengths are reflected at the clamp. This reflection is stored in the form of a look-up table in the pump in order to determine the color of the hose clamp via a relative comparison of the individual colors (RGB,

This means that if other materials are used in the pump, a new lookup table has to be entered. However, it is preferred if the resulting remission spectra remain unchanged in their properties.

| | Normalized | Normalized | Normalized |
|---|---|---|---|
| | remission for blue | remission for green | remission for red |
| | (main wavelength | (main wavelength | (main wavelength |
| Color | 466 nm) | 518 nm) | 629 nm) |
| Magenta | [13.7% . . . 18.1%] | [8.3% . . . 10.1%] | [52.5% . . . 73.0%] |
| Green | [64.5% . . . 86.5%] | [79.3% . . . 100.8%] | [15.5% . . . 19.5%] |
| Gray | [108.0% . . . 126.0%] | [113.5% . . . 133.3%] | [104.5% . . . 122.6%] |
| Red (with IR | [23.3% . . . 28.4%] | [22.1% . . . 26.4%] | [170.7% . . . 208.6%] |
| absorber) | | | |
| White | [226.7% . . . 265.8%] | [231.9% . . . 271.7%] | [217.2% . . . 257.7%] |
| Yellow (with | [35.1% . . . 52.6%] | [106.8% . . . 130.8%] | [113.0% . . . 132.6%] |
| IR absorber) | | | |
| Pink | [66.0% . . . 84.6%] | [39.9% . . . 49.6%] | [55.3% . . . 67.2%] |
| Blue | alternative option | alternative option | alternative option |
| Orange | alternative option | alternative option | alternative option |

It is advantageous if the optical marking is an infrared marker.

It is preferred if the optical window is made of plastic.

It is advantageous if the first light source, the second light source, and the photodetector are arranged on a printed circuit board which is integratable/attachable to the clamping module.

It is preferred if the three LEDs of the first light source have a linear arrangement on the printed circuit board and are arranged at sufficiently small distance to illuminate through the optical window, preferably at a distance of 0.23 mm.

In other words, the combined RGB LED and the IR LED are placed close together so that their illumination is enabled by the optical window. In addition, one RGB LED is installed, which makes it possible to arrange all light sources in alignment, i.e. according to a linear arrangement of the chip layers. The distance between the RGB LEDs is about 0.23 mm. The layout of the circuit board and the selection of the LEDs is chosen to have the smallest possible dimensions and to be integrated into the clamp module of the medical pump/infusion pump.

It is advantageous if the optical window is arranged between the hose clamp/slide clamp and the printed circuit board in such a way that the at least one first light beam and the at least one second light beam hit the hose clamp, in particular the optical marking, through the optical window, and the hose clamp, in particular the optical marking, reflects the at least one first light beam and the at least one second light beam and the reflected light hits the photodetector through the optical window. Furthermore, it is preferred if the arrangement is provided such that the optical window and the arrangement of transmission LEDs and photodetector also ensure that there is no direct light coupling between photodetector and transmission LEDs.

In other words, the arrangement of the light sources, of the photodetector, and of the associated optical window ensures that the reflection area is optimally illuminated and its reflection is received in a bundled manner at the photodetector. The optical window is therefore used to transmit and receive at least one light beam and the reflected light.

Furthermore, the present disclosure relates to a medical pump, in particular an infusion pump, comprising a clamping module according to one of the preceding aspects.

Furthermore, the present disclosure relates to a method for identifying a hose clamp based on at least one color via a measurement algorithm after insertion of the hose clamp into a medical pump, in particular an infusion pump, to determine the type of disposable infusion article used therewith, in particular an infusion hose. A first step (S1) is the measurement of a temperature. In a second step (S2), the ambient light is measured and the ambient brightness is determined. This is followed in the third step (S3) by sequential illumination with red, green, blue and IR light and measurement of the reflection from the hose clamp/ slide clamp. In a final step (S4), the color is evaluated and determined by measured reflection and subsequently compared with an evaluation table stored in the medical pump.

In other words, after inserting the hose clamp, the following is performed:

temperature measurement;

measurement of the ambient light (dark correction) and from this determination of the ambient brightness;

sequential measurement of the reflection on the hose clamp/ slide clamp when it is successively illuminated with, red, green, blue, infrared (IR) light;

the color is determined by evaluating the measured information (separate individual color measurement) and then comparing it with an evaluation table stored in the infusion pump. The evaluation table (lookup table) enables the color to be assigned on the basis of the measured values (remission values).

It is preferred if calibration data are available in a microprocessor, which are used to initialize the measurement algorithm, and the evaluation table has an optical characterization of the hose clamp/slide clamp used with regard to its reflection and fluctuations in the measurement can be compensated via a value range stored therein.

In other words, it is provided that the measured remission spectra are included in the calculation of the single color measurements. The measured values are available as so-called ‘counts’ and are ultimately the analog-to-digital measured values of the photodetector. The measurements are furthermore dependent on the calibration data (white balance), the ambient temperature, and the dark value (ambient light influence). This results in the following calculation basis.

An input requirement is the availability of the calibration data, preferably including the temperature measurement. The calibration data is required for the ‘initialization’ of the measurement algorithm. The calibration data generate a reflection coefficient via a white standard, which is used in the calculation and is dependent on temperature and light intensity in the overall approximation.

Furthermore, the following measured values are recorded. A first measured value is the temperature measurement (Tmess) and a second measured value is the illumination of the hose clamp by individual colors (RGB). The second measured value concerns analog/digital values per color. In a further measured value the dark value is recorded, which describes a measured value that is applied to the photodetector without illumination of the LEDs. It is also necessary to compensate for light entering the clamping module from the outside during the measurement (corresponds to the ambient light above). Thereupon, a correction of the measurement signal with respect to the dark value takes place. The measurement signals themselves are the received data at the photodetector at a measurement time for each individual color. This is followed by a temperature compensation of the measured values via a polynomial that approximates the temperature characteristics of the LEDs. This means that the LEDs and their luminous flux behave differently for different temperatures.

Therefore, the course of this temperature degradation is approximated for each color via a polynomially fitted temperature curve of the LED colors (3rd degree polynomial). Based on this, a reflectivity calculation is performed for each color, which is normalized to the calibration data. These reflectivity values are compared with the minimum and maximum values stored in the lookup/evaluation table. The comparison takes into account additional, possible contamination, which is why the relative ratio of the measurement and evaluation table value is considered in the color assignment. Here, either all measured values of the four colors can be included in the color determination. Alternatively, it is also conceivable to select the intensity/amplitude of the scaling factor if there is no clear color assignment.

The lookup table is generated and measured with the material qualification of the hose clamp/ slide clamp. It contains the optical characterization of the material used with regard to their reflection of the colors red, green, blue and IR. The evaluation table includes a range of values (min./max. value) in order to be able to compensate for fluctuations in the color measurement. Further information on the wavelengths has already been described above. The introduction of an IR component is necessary to distinguish the colors red and yellow from other colors with identical reflectance properties (remission spectrum). Without the added IR markers, which are homogeneously incorporated into the hose clamp/slide clamp, the remission spectra would differ in amplitude, but the actual course and the ratio of all colors to each other would be identical. Clear identification would therefore not be possible.

In summary, the measurement algorithm is based on the idea of illuminating the hose clamp/slide clamp inside the infusion pump with different light sources and determining the color/colors of the clamp via the reflecting light. To do this, however, it is necessary to calibrate the optical system at least once in order to compensate for the influences of the installation situation (here, for example, the tolerance of the installation position) and the tolerances of the electronic components. The calibration generates an offset value pair which is used in the measurement algorithm for compensation.

### BRIEF DESCRIPTION OF THE DRAWINGS

FIG. 1 is a representation of a clamping module with a printed circuit board according to the present disclosure;

FIG. 2 is a representation of a vertical section through the clamping module as shown in FIG. 2 according to the present disclosure;

FIG. 3 is a representation of the structure of a printed circuit board according to the present disclosure;

FIG. 4 is a representation of an interior view of the clamping module as seen from a clamping side according to the present disclosure;

FIG. 5 is a representation of an exterior view of the clamping module as seen from the printed circuit board side according to the present disclosure;

FIG. 6 is a schematic representation of a hose clamp; and

FIG. 7 is a representation of a block diagram of a color measurement and its implementation within an infusion pump according to the present disclosure.

### DETAILED DESCRIPTION

The following describes configuration examples of the present disclosure based on the accompanying figures.

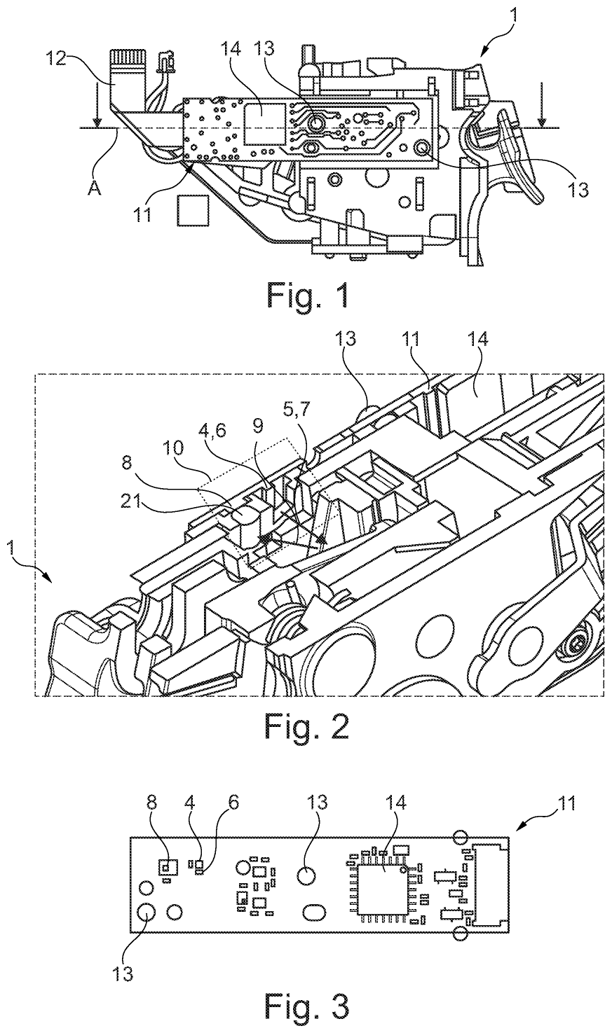

FIG. 1 is a representation of a clamping module 1 with a printed circuit board 11 according to the present disclosure. The clamping module 1 is designed to be insertable/integratable into a medical pump, in particular an infusion pump. A printed circuit board 11 is attached to or integrated in the clamping module 1. The upper side of the printed circuit board 11 faces the clamping module 1. The printed circuit board 11 is fixed to the clamping module 1 with at least one fastening means 13, according to FIG. 1 with two fastening means 13. On the printed circuit board 11, a first part of an electro-optical system is placed and a second part of the electro-optical system is part of the clamping module 1. This is described in FIGS. 2 and 3 in more detail.

In addition, FIG. 1 shows a ribbon cable 12 that is plugged into a main board of the medical pump. Furthermore, it can be seen on the printed circuit board 11 where a microprocessor 14 is arranged. Details of this are described in more detail for FIG. 3.

FIG. 2 is a representation of a vertical section through the clamping module 1 as shown in FIG. 1 according to the present disclosure. In FIG. 1, a sectional axis A is drawn. Based on the sectional axis A, FIG. 2 is shown in a top view. Thus, in FIG. 2, the printed circuit board 11 is shown on the side of the clamping module 1 facing away from the viewer. In connection with the printed circuit board 11, the one fastening means 13 (screw, rivet, etc.) can be seen, which serves to fix the printed circuit board 11 to the clamping module 1, preferably via screws or soldering.

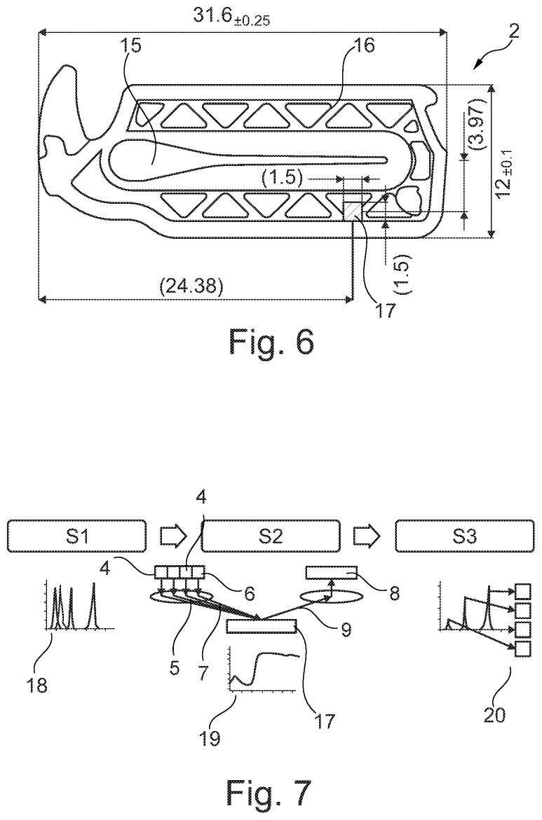

FIG. 2 further shows that a first light source 4, a second light source 6 with corresponding transmitting optics, and a photodetector 8 with corresponding receiving optics are arranged on the printed circuit board 11. The optical window 10 is arranged between the first light source 4, the second light source 6, and the photodetector 8. The optical window 10 is configured in such a way that at least a first light beam 5 and a second light beam 7 are bundled and pass through the optical window 10 onto the hose clamp 2 (not shown here, see FIG. 6) to be inserted. Furthermore, the optical window 10 consists of two plastic lenses and is designed so that a light 9 reflected by the hose clamp 2 is in turn bundled and received by the photodetector 8 on the printed circuit board 11.

The at least one first light beam 5 and the second light beam 7 is shown in FIG. 2 as an arrow pointing away from the printed circuit board 11. The reflected light 9 is shown in FIG. 2 as an arrow in the direction towards the printed circuit board 11. The optical window 10 forms such an opening in the housing of the clamping module 1 to allow the light beams 5 and 7 as well as the reflected light 9 to pass through.

Against this background, the arrangement of the printed circuit board 11, in particular with respect to the first light source 4 and to the second light source 6 as well as to the photodetector 8, to the clamping module 1 and to the hose clamp to be inserted into the clamping module 1 is clearly defined.

FIG. 3 is a representation of the structure of a printed circuit board 11 according to the present disclosure. The printed circuit board 11 is shown on the rear side. The printed circuit board in FIG. 3 shows the arrangement of the first light source 4, the second light source 6, and the photodetector 8 with respect to each other. Here, it can be seen that the first light source 4 and the second light source 6 are arranged next to each other in the width direction of the printed circuit board 11. The photodetector 8 is arranged with respect to the first light source 4 and the second light source 6 in the longitudinal direction thereof.

Furthermore, FIG. 3 shows the two fastening means 13 as well as a microprocessor 14, which is also arranged on the printed circuit board 11.

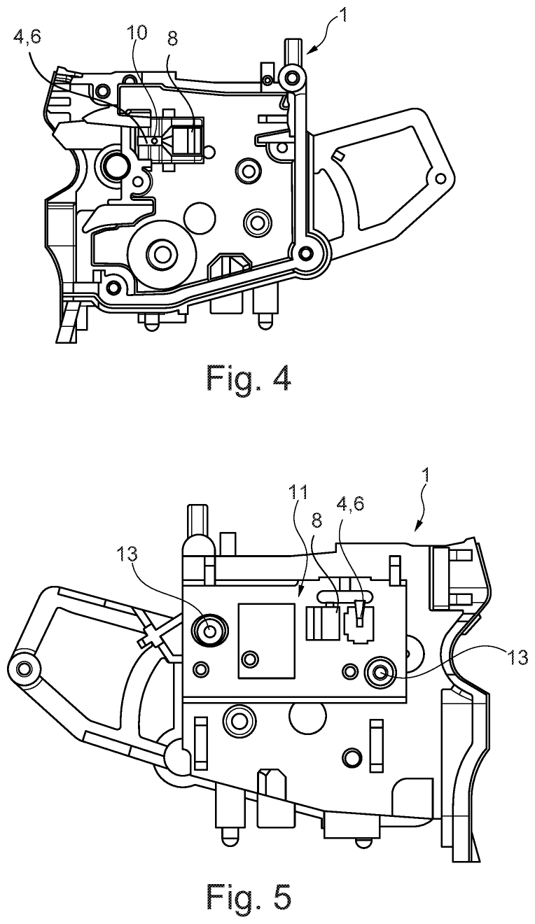

FIG. 4 is a representation of an interior view of the clamping module 1 as seen from a clamping side according to the present disclosure. In other words, FIG. 4 shows a section through the clamping module 1. Here, the printed circuit board is arranged on a side not facing the viewer and the cut is made such that the optical window 10 is visible. The clamping module 1 therefore shows the optical window 10, which consists of two plastic lenses and behind which the photodetector 8 and the first light source 4 as well as the second light source 6 are arranged.

FIG. 5 is a representation of an exterior view of the clamping module 1 as seen from the side of the printed circuit board 11 according to the present disclosure. In FIG. 5, the optical window 10 and the ‘receptacle’ of the printed circuit board 11 for attachment to the clamping module 1 are shown schematically. That is, FIG. 5 is shown from the other side in contrast to FIG. 4. In addition, the fastening means 13 are shown. On the depicted outside of the clamping module 1 for receiving the printed circuit board 11, the optical windows 10 or recesses can be seen through which the light beam 5 and 6 of the first light source 4, of the second light source 6, and of the photodetector 8 are depicted. The light source 4 and 6 as well as the photodetector 8 are not shown directly, but only their position when the printed circuit board 11 is inserted into the receptacle.

FIG. 6 is a schematic representation of a hose clamp 2. The hose clamp 2 has a tapered elongated hole 15 through which an infusion hose extends. The infusion hose is not closed at one end of the elongated hole 15 and is compressed when it is pushed to the other end of the elongated hole 15. When the disposable article is inserted, the hose clamp 2 is in an open state. In this state, the hose clamp 2 is inserted into the clamp module 1. When a front flap of the medical pump is closed, the hook shown on the hose clamp 2 engages an eyelet of the front flap (and at the same time, a pump-side clamp is closed to prevent free flow). When opening the front flap, the hose clamp 2 pulls over the infusion hose and thus closes the passage. In parallel, the medical pump 1 opens its pump-side clamp. Both together continue to prevent free flow. The reference sign 16 relates only to a reinforcement of the hose clamp 2 itself. Near the narrow end of the elongated hole 15, the hose clamp 2 has a measuring point 17. The measuring point 17 is provided and configured to reflect the at least one first light beam 5 and the at least one second light beam 7 and to return the reflected light 9 to the photodetector 8.

Furthermore, FIG. 6 shows the dimensions of the clamping module 2. The total length (31.6 mm) shown in FIG. 6 corresponds to a total length for identifying that the hose clamp 2 is inserted into the clamping module.

FIG. 7 is a representation of a block diagram of a color measurement and its implementation within an infusion pump according to the present disclosure. In a first step Si the sequential illumination of the sample or respectively of the measuring point 17 of the hose clamp 2 takes place.

The sequential illumination results in illumination spectra according to the representation to reference sign 18. Hereby, the measuring point 17 is hit by the at least one first light beam 5 in the colors red, green and blue starting from the first light source 4 and by the second light beam 7 starting from the second light source 6. The measuring point 17 reflects in a step S2 and the reflected light 9 hits the photodetector 8, which records the spectral properties of the material according to reference sign 19. In a third step S3, a remission spectrum generates a photocurrent in the photodetector 8. The resulting signal is output in an evaluation spectrum according to reference sign 20.

It is preferred to use thermoplastic based on polyolefin or polyoxymethylene with the following properties:

E-modulus: >1000 MPa

Yield strength: >27 MPa

Friction coefficients based on pen-writing friction test in accordance with ASTM G115 and DIN EN ISO 7148-2

| Size | Value |
|---|---|
| Structure | test pin: slide clamp |
| | test plate: hose material |
| Measurement | 20 cycles |
| Cycle | 1. feed motion (tR = 2 s) |
| | 2. standstill (tP = 2 s) |
| | 3. reset movement (tR = 2 s) |
| | 4. standstill (tP = 2 s) |
| Friction path | 15 mm |
| Sliding speed | 450 mm/min |
| Surface pressure pN | 2.7 +/− 0.1 MPa |
| Hose material | PVC-P and TPU |

We claim:

1. A clamping module of or for a medical pump, which forms or has an electro-optical system for detecting a hose clamp, having at least a predetermined color and/or an optical marking, of a disposable infusion article insertable into the clamping module, having the following system components:
a first light source for transmitting at least a first light beam having a first frequency;
a second light source for transmitting at least a second light beam having a second frequency, wherein the second light source is formed differently from the first light source and/or the first frequency is formed differently from the second frequency;
a photodetector for receiving light reflected from the disposable infusion article; and
an optical window for focusing the first light beam and/or the second light beam on the disposable infusion article as well as the light reflected from the disposable infusion article.

2. The clamping module according to claim 1, wherein the first light source is an RGB LED, and the second light source is an IR LED, wherein the first light source comprises three LEDs which are configured to output the first light beam as a red, a green and/or a blue first light beam.

3. The clamping module according to claim 1, wherein the predetermined color and/or the optical marking comprises an optical marker, and wherein the optical marker is an infrared marker.

4. The clamping module according to claim 1, wherein the optical window is made of plastic.

5. The clamping module according to claim 1, wherein the first light source, the second light source, and the photodetector are arranged on a printed circuit board which is integratable/attachable to the clamping module.

6. The clamping module according to claim 5, wherein the first light source comprises three LEDs having a linear arrangement on the printed circuit board and are arranged at a distance to illuminate through the optical window.

7. The clamping module according to claim 5 , wherein the optical window is arranged between the hose clamp and the printed circuit board in such a way that:
the first light beam and the second light beam hit the hose clamp through the optical window,
the hose clamp reflects the first light beam and the second light beam, and
the light reflected from the disposable infusion article hits the photodetector through the optical window.

8. A medical pump comprising the clamping module according to claim 1.

9. A method for identifying a hose clamp based on a color via a measurement algorithm after insertion of the hose clamp into a medical pump, the method comprising the steps of:
measuring a temperature;
measuring an ambient light;
determining an ambient brightness;
sequentially illuminating the hose clamp with red, green, blue and IR light;
measuring a reflection of light from the hose clamp; and
evaluating and determining the color by measuring the reflection of light and comparing a measurement of the reflection of light with an evaluation table stored in the medical pump.

10. The method according to claim 9, wherein calibration data are available in a microprocessor, which are used to initialize the measurement algorithm, and the evaluation table has an optical characterization of the hose clamp used with regard to the reflection of light, and fluctuations in the measurement of the reflection of light are compensated via a value range stored in the microprocessor.

11. A hose clamp for insertion into the clamping module according to claim 1,
wherein the predetermined color of the hose clamp is configured and provided to be usable by an algorithm, and/or
the optical marking is configured and provided to separate similar colors and/or reflection spectra.
